(19) United States
(12) Patent Application Publication
(10) Pub. No.: US 2024/0074975 A1
(43) Pub. Date: Mar. 7, 2024

(54) NANOVESICULES DERIVING FROM BIOLOGICAL PLANTS AS NATURAL CARRIERS OF PHYTO-COMPLEXES FOR NUTRACEUTICAL, COSMETIC AND REGENERATIVE USE

(71) Applicant: EXO LAB ITALIA, S.r.l., Pescara (IT)

(72) Inventors: Mariantonia LOGOZZI, Pescara (IT); Stefano FAIS, Pescara (IT)

(73) Assignee: EXO LAB ITALIA, S.r.l., Pescara (IT)

(21) Appl. No.: 18/272,531

(22) PCT Filed: Jan. 24, 2022
(86) PCT No.: PCT/IB2022/050587

§ 371 Date: Jul. 14, 2023

(30) Foreign Application Priority Data
Jan. 25, 2021 (IT) 102021000001343

(51) Int. Cl. A61K 9/127; A61K 8/14; A61K 8/64; A61K 8/66; A61K 8/67; A61K 31/375; A61K 36/185; A61K 36/22; A61K 36/752; A61K 38/06; A61K 38/44; A61P 17/02; A61P 39/06; A61Q 19/08

(52) U.S. Cl. CPC A61K 9/127; A61K 8/14; A61K 8/64; A61K 8/66; A61K 8/676; A61K 31/375; A61K 36/185; A61K 36/22; A61K 36/752; A61K 38/063; A61K 38/44; A61K 38/446; A61P 17/02; A61P 39/06; A61Q 19/08

(57) ABSTRACT

Nanovesicles derived from plants of biological origin, comprising a lipid membrane and within the lipid membrane at least one bioavailable antioxidant substance, and use of the same nanovesicles for nutraceutical, cosmetic and regenerative use.

### BACKGROUND OF THE INVENTION

### 1. Field of the Invention

The present invention relates to nanovesicles deriving from biological plants and their use as natural carriers of phyto-complexes, in particular for nutraceutical, cosmetic and regenerative use. These vesicles contain at least one bio-available antioxidant substance within the lipid membrane. Background art

### 2. Brief Description of the Prior Art

As known, in recent years, scientific research on extracellular vesicles (EV) has grown considerably, leading to the production of an enormous amount of data. These vesicles take an active part in numerous functions involved in the complex and integrated regulation of our organism, but also of most living species (Yanez-M6 et al., 2015). Recent evidence has also proposed EVs as a natural vehicle for therapeutic molecules (Fais, 2016; Lener et al., 2015). In this context, while there are numerous data obtained from human and animal cells, there is little information regarding extracellular vesicles (EVs) coming from food of various origins, ie from foods. The presence of exosomes (or endosomally derived vesicles) in food was included in the FAO/INFOODS database (FAO, 2014), which was called “FoodEVs”. In reality, information on Food EVs is limited to four FAO groups, namely milk, starchy roots and tubers, nuts and seeds, and fruit. However, previous studies have suggested that nanovesicles deriving from plant cells could be very similar to exosomes of human and animal origin (Regente 2009); moreover, preliminary data have shown that nanovesicles derived from edible fruit and vegetables (grapes, grapefruit, ginger and carrots) are able to perform anti-inflammatory activity (Wang B 2014; Ju 2013). On the other hand, it has also been shown that the aqueous compounds and/or extracts of different plant varieties exert anti-proliferative and antitumor activity (Wang L 2014; Manthey 2001; Benavente-Garcia 0 2008; Blagosklonny 2005). To date, the only scientific evidence regarding fruit as a source of EVs concerns the juice of Citrus limon L. (family Rutaceae); the study shows that lemon juice contains nanovesicles with characteristics similar to exosomes (Raimondo 2015); in the same work it is shown that EVs deriving from lemon have anti-neoplastic activity in vitro and in vivo.

Citrus fruits are the most relevant product in the world in the agricultural market and are one of the main sources of vitamin C (Vit. C). Among the various types of fruit, citrus fruits contain the highest amount of carotenoids and a wide range of secondary compounds with fundamental nutritional properties. In general, citrus fruits are a fundamental source of nutraceutical compounds with antioxidant and anti-inflammatory, but also anti-tumor activity (Granger 2018). Of great importance are recent data that suggest the possibility that the complex of bio-actives is contained in the nanovesicles deriving from citrus fruits in packaged form, therefore in the form of a bio-complex, and therefore not of single elements, thus suggesting that in nature bio-actives are not available as single supplements but as complexes within exosome-like nanovesicles. As a matter of fact, nanovesicles similar to exosomes of Citrus limon L. (EXO-CLs) have shown antioxidant capacity and therapeutic activity, with potential use in the treatment of numerous diseases (Baldini 2018). In vitro and in vivo studies have also shown that nanovesicles deriving from grapefruit (grapefruit-derived nanovectors, GNVs) carry a variety of therapeutic agents, including chemotherapy drugs, DNA expression vectors, siRNAs and proteins of various types. It is important to underline that GNVs can be modified to be directed towards specific cellular targets, therefore with a more direct use in medicine to help the whole organism to react in the best way to pathological insults. It is also desirable that nanovesicles deriving from edible vegetables and fruit can be used as natural carriers of therapeutic molecules with significant advantages such as: (i) absence of detectable toxicity; (ii) constitutive presence of a great variety of therapeutic bio-actives and (iii) possibility of producing large quantities of them in a sustainable way for humans and the environment.

The use of nanovesicles deriving from plants deriving from non-GMO, organic and biodynamic cultivation is not known in the state of the art. A recent study by the applicant has shown that nanovesicles can concentrate toxic substances eliminated from cells. Using human primary cells it has been shown that macrophages “scavenge” by eliminating, for example, nanoparticle gold through exosomes (Logozzi et al European Journal of Pharmaceutics and Biopharmaceutics 137 (2019) 23-36), which in addition to paracrinally transmitting the toxic substance can diffuse it by the whole organism through the bloodstream. It is therefore very likely that the same happens in plants, when grown intensively through the use of toxic substances or through GMO technologies. In fact, nanovesicles located in the paramural space of plants, are similar in structure and function to those isolated from mammals

(An et al., 2007); and are known as edible nanovesicles of plant origin (PDEN: Plant Derived Edible Nanovesicles). In nature, nanovesicles play a key role in cell-cell communication, both within an organ or apparatus, and at a distance; such phenomenon operating both within the same species and between different species.

Nanovesicles of both plant and animal origin are natural carriers of proteins, lipids and nuclear components, but with variations from species to species. It is known that plants contain intrinsic and unique compounds with physiologically relevant bioactivities, and therefore also the molecular content of the nanovesicles varies according to the plant from which they are released (Zhang et al. 2016). In their function of communication between cells and organs, the nanovesicles have the ability to transfer their contents inside target cells through membrane-membrane fusion, and it is therefore very likely that nanovesicles of plant origin can transfer their contents into human cells, using the same mechanism.

In conclusion, nanovesicles deriving from plants constitutively contain bioactive molecules potentially effective in the treatment of vitamin deficiencies or in any case potentially usable in health management, both in a preventive regime and in the treatment of some pathologies .

As is also known, plant nanovesicles can also be concentrated and used as nanocarriers of bioactive compounds other than their intrinsic compounds.

Furthermore, the nanovesicles of plant origin, obtained by modifying standard methods, contain bioactives complexed together and capable of having beneficial effects both in vitro and in vivo.

The natural bioactives known and currently on the market show these criticalities:
extraction with alcohol, hydroalcoholic solutions or dry glycerine macerate; chemical synthesis; low bioavailability;
sensitive to oxidation;
lower stability and very fast pharmacokinetics after about 2 hours they are no longer in circulation and are eliminated in the urine; at the time of digestion low absorption, therefore consumption of large quantities to have a minimal effect .

However, the use of nanovesicles extracted from plants coming from biological crops and containing within the lipid membrane at least one bio-available antioxidant substance, and their use as natural carriers of phytocomplexes, in particular for nutraceutical, cosmetic and regenerative use, is however not known.

### SUMMARY OF THE INVENTION

Aim of the present invention therefore concerns vesicles extracted from biological plants and their use as natural carriers of phytocomplexes, in particular for nutraceutical, cosmetic and regenerative use.

The nanovesicles extracted from plants of biological origin, are obtained with standard methods and contain bioactives complexed together and able to have beneficial effects both in vitro and in vivo. The method through which nanovesicles from various fruits have been obtained, comprises repeated cycles of centrifugation and ultracentrifugation, as required by the internationally shared standard procedures (ISEV)

Advantageously, the nanovesicles were extracted from juice squeezed from biological vegetables, such as for example Citrus paradisi, Citrus lemon (L.), Citrus reticulata, Citrus bergamia, Actinidia chinensis, Mangifera indica, Carica papaya linn, Citrus sinensis, Malus domestica.

Advantageously, these nanovesicles of plant origin contain a high concentration of bio-available antioxidant vitamins such as ascorbic acid. The main limitation of all antioxidants available on the market of enzymatic and non-enzymatic nature, is the low level of bioavailability caused by the high instability of the molecules which are both photosensitive and thermolabile and which require oxidation in order to be absorbed by the intestinal mucosa.

These water-soluble vitamins are involved in multiple functions of our body, such as the formation of collagen, the absorption of iron, cellular respiration and the strengthening of the immune system. Regarding their antioxidant capacity, vitamin C allows the restoration of vitamin E from free radicals produced during the peroxidation of fats. Vitamin C is found mainly in citrus fruits such as oranges, grapefruits, mandarins, lemons, cedars, etc., and in all acidic fruits such as kiwis, strawberries, currants and raspberries. Good quantities are also present in red chili, tomatoes and to a lesser extent in green vegetables (turnip broccoli, cabbage, peppers, asparagus, etc.); in contact with air and light, vitamin C undergoes rapid oxidation, considerably reducing its antioxidant capacity.

Advantageously, the bioactive antioxidants contained in the nanovesicles from organic farming:
they are natural nanovesicles designed by nature to be a perfect tool for intercellular communication;
their lipid membranes protect the bioactive at the inside from acidity and thermal shock;—they are directly absorbed by the cells as the lipids are highly fusogenic (membrane/membrane fusion), pouring their content directly into the cells;
due to their lipid composition they pass the blood brain barrier and the placenta;
they are not easily attacked by the immune system;
they have a great scalability at an industrial level;
they are not toxic as they derive directly from fruit and vegetables, from biological farming.

Therefore, according to a first aspect of the present invention, nanovesicles extracted from plants of biological origin are defined, comprising a lipid membrane and containing within the lipid membrane at least one bio-available antioxidant substance, as specified in the attached independent claim.

According to a further aspect, the use of such nanovesicles is defined as nutraceutical, cosmetic and regenerative, as specified in the independent claims for use. The dependent claims outline particular and further advantageous aspects of the invention.

### BRIEF DESCRIPTION OF THE DRAWINGS

These and other advantages of the invention will now be described in detail, with reference to the attached drawings, which represent an exemplary embodiment of the invention, in which: FIG. 1 shows the distribution of nanovesicles isolated from citrus juice, according to the present invention;

FIG. 2 shows the difference in quantitative production of nanovesicles from biological and conventional agriculture;

FIG. 3 shows the comparison between the profile of the vesicles treated with the lysant (Tris HCl IM, pH 8.6) and the profile of the same vesicles before the treatment (exo in PBS);

FIG. 4 shows the comparison between the ascorbic acid content of the vesicles treated with the lysant and those not treated with the lysate;

FIG. 5 shows the analysis of cell mortality through the “Trypan Blue Assay” of the treatment with plant nanovesicles on cell cultures of keratinocytes and fibroblasts both with single treatment and with two subsequent treatments;

FIG. 6 shows the cell regeneration after treatment of cells plated with nanovesicles and with ascorbic acid;

FIG. 7 shows the increase in the production of type I collagen after treatment of human keratinocytes and fibroblasts with nanovesicles;

FIG. 8 shows the toxicity and efficacy graphs of the administration of nanovesicles in mouse models;—FIG. 9 shows the increase in the heavy and light chains of the immunoglobulins in mice treated with a first composition containing mixtures of nanovesicles, compared to those treated with H2O2 alone;

FIG. 10 shows the proliferation capacity in the bone marrow and spleen of mice treated with a first composition containing mixtures of nanovesicles, compared to those treated with H2O2 alone;

FIG. 11 shows the telomere length of ovarian germ cells obtained from prematurely aged mice following treatment with H2O2 and treated or untreated with a second composition containing mixtures of nanovesicles;

FIG. 12 shows levels of oxidizing substances (ROS) and lipid peroxidation present in the blood of mice treated and not treated with the second anti-aging composition.

### DETAILED DESCRIPTION OF THE PREFERRED EMBODIMENTS

According to the present invention and on the basis of the accompanying Figures, the nanovesicles have been extracted from vegetables of biological kind. With a biological agriculture an agriculture is meant that uses a cultivation technique and a way of producing food that respects natural life cycles. That is, without the use of chemical pesticides, synthetic fertilizers, antibiotics and other substances which are subject to strict restrictions. In addition, the crops are rotated so that the resources on site are used efficiently; local resources are exploited, such as manure for fertilizer or feed produced on the farm. Furthermore, biological agriculture by definition does not use genetically modified organisms (GMOs). On the contrary, plant and animal species resistant to diseases and adapted to the environment are used. For this purpose, techniques such as the safeguarding of beneficial insects, antagonists of parasites, are used; rustic, more resistant plants are chosen; mulching is practiced, which consists in covering the ground with hay or fresh grass in order to protect it from temperature changes and hinder the growth of weeds; green manure is used, that is the sowing of some plants (clover, vetch, cress, lamb's lettuce, spinach, rapeseed and so on) which, once in bloom, are buried to fertilize the soil and protect it from erosion; crop rotation is practiced, which consists in alternating the cultivation of plants that improve the fertility of the soil, for example by enriching it with nitrogen, with plants that deplete it, subtracting nutritional elements; manure and organic fertilizers are used such as compost, a mixture of earth, plant remains, wood ash and anything else that exists on the farm that is biodegradable and unpolluted. The fruits used are obtained from Italian companies specialized and certified for organic cultivation, carefully washed with water and bicarbonate and subjected to mechanical extraction. The methodology through which the nanovesicles were obtained from the various fruits includes repeated cycles of centrifugation and ultracentrifugation, as required by the internationally shared standard procedures (ISEV). The juice thus obtained is filtered using a 100 micron pore filter. Subsequently, the juice is centrifuged at 500×g for 10 minutes, 2000×g for 20 minutes and 15000×g for 30 minutes, respectively. The supernatant was then centrifuged at 110, 000×g for 90 minutes, using a fixed angle rotor, the pellet was then suspended in phosphate-buffered saline (PBS) or in ultra-filtered water.

Subsequently, quantifying of the nanovesicles is performed by “Bradford assay” (Pierce, Rockford, IL, USA) and “Nanoparticle Tracking Analysis” (NTA). The NTA analysis demonstrated the integrity of the vesicles and allowed the evaluation of the concentration, size and distribution of the vesicles derived from the extracted juice. In particular, the dimensions of the vesicles obtained are comprised between 80 nm and 200 nm, thus demonstrating the high number of nanovesicles in the fruits examined (as shown in FIG. 1). Tests carried out have shown that the vesicles obtained from fruit from biological and biodynamic agriculture are better than those obtained from intensive agriculture vegetables and contain at least one bio-available antioxidant substance inside the lipid membrane. By bioavailability the degree and speed is meant in which the active form of a drug (i.e., the drug itself or one of its metabolites) reaches the systemic circulation, thus acquiring the ability to access its site of action. But what is more important is that the administered drug fraction reaches the systemic circulation without undergoing any chemical modification with respect to the total quantity administered, and therefore is totally available. This is a paradigm referring to drugs in the strict sense but which must necessarily also refer to supplements and nutraceuticals. Even if the definition refers to a complex system, such as any animal organism, including humans, a simpler level of bioavailability can also be established in culture ‘when the presence of, for example, a vitamin or a phytocomplex in its native form (e.g. not oxidized), is tested.

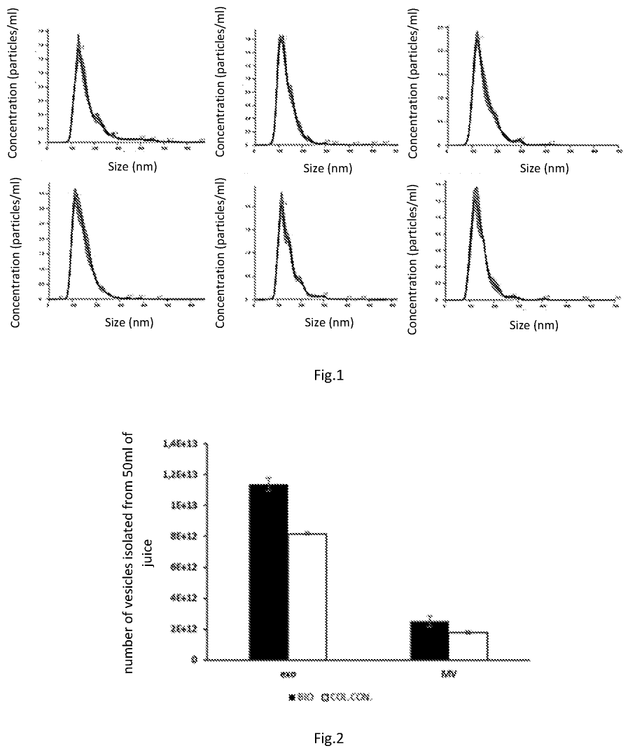

The independent experiments, as shown in FIG. 2, in which the number of nanovesicles obtained from biologically derived fruit was compared with the number of nanovesicles obtained from fruit from intensive cultivation, have shown that biologically derived fruit provides a yield in nanovesicles clearly greater than that deriving from conventional cultivation. The tests carried out have shown that the vesicles obtained from fruit from biological and biodynamic agriculture have antioxidant activity. Through further experiments and using a commercial kit (PAO kit) to measure the total antioxidant activity of our both micro and nanovesicle preparations, as shown in table 1, the results showed a marked dose-dependent antioxidant capacity in the nanovesicle preparations derived from fruit, so demonstrating that within the fruit nanovesicles there were bioactives with marked antioxidant activity. Table 1 shows the data obtained from the analysis of various samples of vesicles isolated from fruit.

TABLE 1

| | Average ± st. error | |
|---|---|---|
| Average | 158.8 ± 10.3 | nm |
| Mode | 121 ± 4.2 | nm |

| | |
|---|---|
| Number of exosomes | 2.7 × 10¹³ ± 2.4 × 10¹² |
| isolated from 50 ml of | |
| juice mix from citrus | |
| and papaya, kiwi and | |
| mango | |

FIG. 2 shows the differences in vesicle production in biological (BIO) and conventional (COL. CON.) grapefruit samples, in which the micro- and nanovesicles isolated from the juice of the grapefruit mentioned above were analyzed with NTA. As can be seen in FIG. 2, starting from the same volume of juice (50 ml), many more vesicles are obtained from the BIO culture samples (exo BIO: 1.14×1013±4.18×1011; MV BIO 2.5×1012±9.3×1010) compared to those from conventional cultivation (exo COL. CON. 8,2×1012±3,5×1011; MV COL. CON. 1, 8×1012±9, 8×1010).

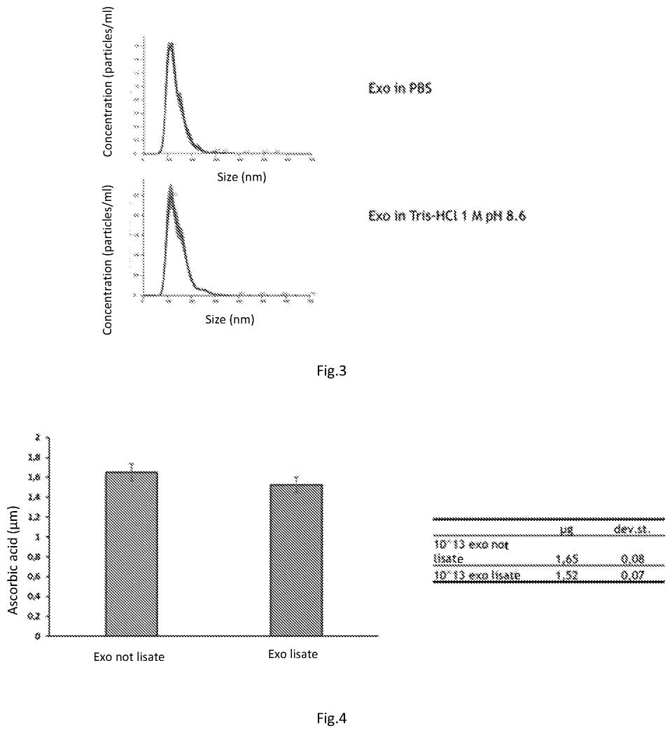

As shown in FIGS. 3 and 4, experimental tests have also shown that the nanovesicles contain vitamin C at their inside, which is protected by the lipid membrane and therefore from degradation when exposed to air and light. Advantageously, vitamin C contained in the vesicles is present in a natural formulation that can be used directly to its maximum potential.

The antioxidants were measured with commercial kits by determining the quantity present in a range of nanovesicles (from 106 to 1013) measured through the Nanoparticle Tracking Analysis (NTA) technique.

The mean concentrations are shown in Table 2 below.

TABLE 2

| | | |
|---|---|---|
| Total antioxidant capacity | 1758 ± 19 | mM |
| Superoxide dismutase type | 542 ± 11 | U/ml |
| 1 (SOD-1) | | |
| Catalase | 1317 ± 397 | mU/ml |
| Glutathione | 551 ± 1.3 | μM |
| Ascorbic acid | 4.6 ± 0.8 | mg |

Furthermore, nanovesicles extracted from biological plants contain bioactive substances such as ascorbic acid and other enzymatic antioxidants protected by the lipid membrane.

To evaluate the ability of the exosomal membranes to protect their internal antioxidant components (such as ascorbic acid), membrane lysis tests were carried out using both chemical (Tris HCl IM, pH 8.6) and physical lysants (50 kHz ultrasonication for 2 minutes). As shown in FIG. 3, vesicles treated with IM tris HCl (pH 8.6) used for plant cell lysis were analyzed and distribution profile at NTA was measured.

The profile of the vesicles treated with the lysant (Tris HCl IM, pH 8.6) was then compared with the profile of the same vesicles before treatment (exo in PBS). As can be seen in FIG. 3, the two profiles coincide, demonstrating that the lysant is unable to break the exosomal membranes.

For further testing the resistance of the membranes and their ability to protect the antioxidant components contained in them, the nanovesicles were ultrasonicated and then the ascorbic acid content in lysed and nonlysed exosomes was evaluated. The same number of vesicles (1013 vesicles) was used for this analysis. The results in FIG. 4 show that the ascorbic acid content does not vary much between lysed and non-lysed sample, further demonstrating the great resistance of the exosomal membranes.

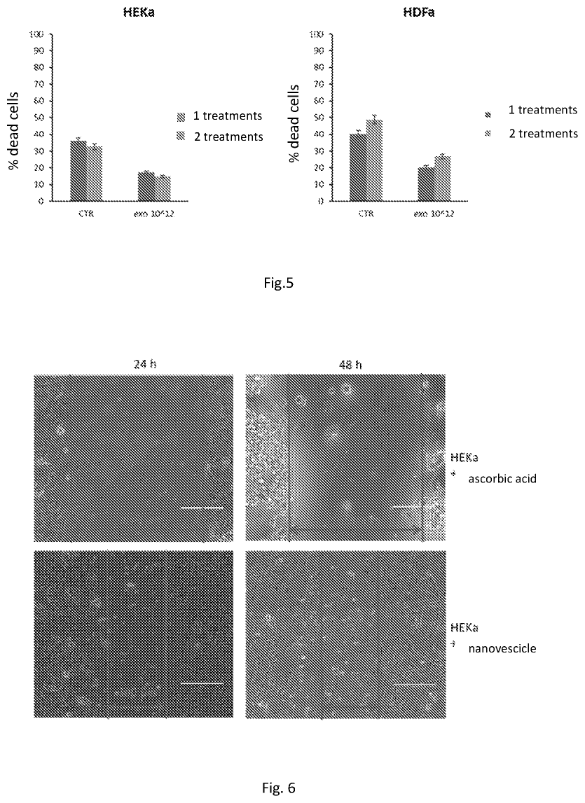

Further tests are shown in FIG. 5, which demonstrate that the nanovesicles are not toxic in the treatment of human cells such as keratinocytes and fibroblasts. The analysis of cell mortality through the “Trypan Blue Assay” has shown that the treatment with plant nanovesicles on cell cultures of keratinocytes and fibroblasts does not induce cytotoxicity both with single treatment and with two subsequent treatments. The nanovesicles induce cell regeneration after treatment (Wound Healing Assay). The keranocyte cells were plated and a wound to the cell monolayer was induced after 18 hours. One sample was treated with microvesicles and the other with ascorbic acid. Optical microscope images were acquired after 24 and 48 hours (FIG. 6).

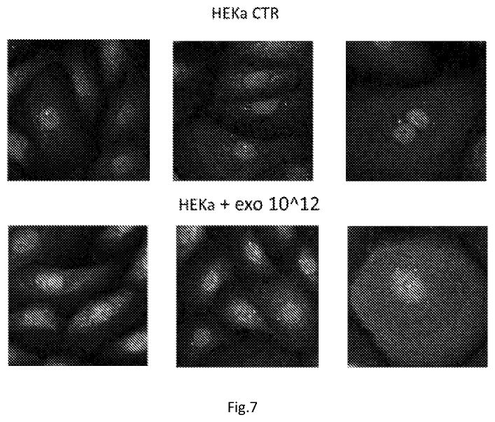

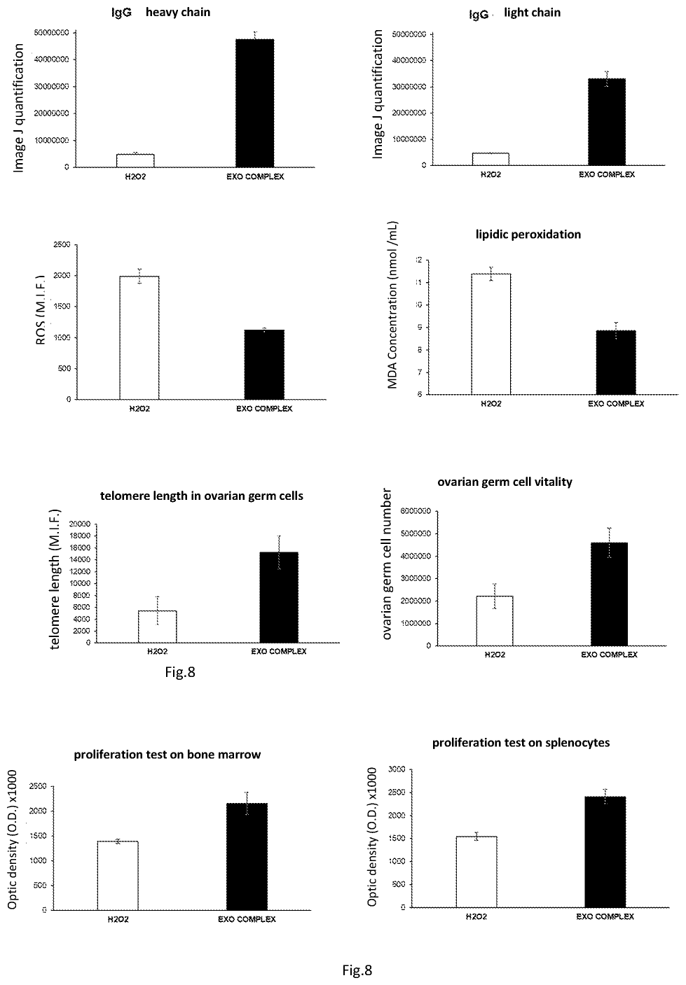

The nanovesicles are able to increase the production of type I collagen after treatment of human keratinocytes and fibroblasts. The test shown in FIG. 7 evaluates the effect of the antioxidants contained in the nanovesicles. The keratinocytes were treated with various doses of vesicles and the presence of type I collagen was detected through Confocal Laser Scanning Microscopy. The images show an evident increase in collagen production (green signal) of HEKa cells (Keratinocytes) 24 hours after treatment with a dose of 10¹² exosomes.

Finally, to verify the ability of nanovesicles to induce an antioxidant reaction and to verify the in vivo toxicity after administration by gavage or intraperitoneum, some experiments were performed in a mouse model (C57B1/6), in which 60 mice were hydrated with water with the addition of H2O2 and subsequently were divided into two groups, in which the one continued to take only water with the addition of H2O2 and the other one received also the nanovesicles.

Mice were tested for some standard parameters (weight, hair luster, etc.) and for systemic levels of oxidants (e.g. ROS, lipid peroxidation, immunoglobulin production)

Furthermore, some molecular parameters of aging were evaluated such as the length of the Telomeres. The results showed that micro-vesicles induce: (i) increased vitality and proliferation of marrow and splenocyte cells; (ii) reduction of ROS in plasma of treated mice; (iii) reduction of malondialdehyde in plasma from treated mice and (iv) elongation of telomeres in ovarian germ cells. In addition, an ex vivo test on bone marrow and splenocyte cells showed increased production of immunoglobulin heavy and light chains in mice treated with microvesicles. An example of a formulation as a nutraceutical includes a range of nanovesicles used from 1×10⁶ to 1×10¹³ derived for example from Citrus lemon, Citrus sinensis, Actinidia chiensis or Carica papaya linn.

An example of a formulation as a cosmetic includes a range of nanovesicles used from I×IO⁵ to I×IO¹² derived for example from Citrus paradisi Mangifera indica or Carica papaya linn.

An example of a formulation as a regenerative includes a range of nanovesicles used from I×IO⁶ to I×IO¹³ derived for example from Citrus lemon, Citrus bergamia or Citrus paradisi.

According to a further aspect of the present invention, new compositions have been prepared comprising nanovesicles extracted from different fruit mixtures. The analysis of the nanovesicles deriving from a large number of certified Italian organic fruits and vegetables made it possible to qualitatively evaluate all the compositions.

Advantageously, each single mixture is intended for different uses. The composition of the mixtures was established in terms of the amount of Superoxide Dismutase Type I, Catalase, Glutathione and Ascorbic Acid. In addition, the total antioxidant capacity (PAO) was evaluated. Advantageously, from the studies and tests carried out, the total antioxidant capacity of the at least one bio-available antioxidant substance for a dose equal to 5×10¹⁰ nanovesicles is greater than 1600 mW.

A first composition, as demonstrated by experimental tests, comprises a mixture capable of inducing immune-stimulation and immune-nutrient effects developed on the basis of the properties of the individual fruits.

The first composition includes a mixture of fruits derived from certified Italian organic cultivations, extracted in the percentages shown in Table 3 below.

TABLE 3
COMPOSITION MIX

| | |
|---|---|
| Citrus Lemon | 18-22% |
| Citrus Sinensis | 18-22% |
| Citrus Sinensis | 9-11% |
| (Magnolopsida | |
| cultivar) | |
| Carica Papaya Linn | 18-22% |
| Mangifera Indica | 9-11% |
| Actinidia Chinensis | 18-22% |

Concentrated nanovesicles are obtained from such fruit mixture, according to the previously indicated extraction procedure.

The choice of the daily dose is defined on the basis of the administration way, and can be for example equal to 5×10¹⁰ nanovesicles (immuno forte exo complex) or 5×10⁹ nanovesicles (immune normal exo complex). The quantification of the antioxidants contained in the dose of 5×10¹⁰ nanovesicles (immune forte exo complex) is shown in Table 4 below.

TABLE 4
Quantification of antioxidants per dose 5 × 10¹⁰ nanovesicles

| | | |
|---|---|---|
| Total antioxidant | 1739.0 ± 130 | mM |
| capacity | | |
| Superoxide dismutase | 556.8 ± 13 | U/ml |
| type I | | |
| Catalase | 1713.3 ± 30 | mU/ml |
| Glutathione | 549.4 ± 15 | μM |
| Ascorbic acid | 32.3 ± 0.1 | μg |

The immunostimulating effect of said first composition was verified with in vivo experiments in a mouse model in which immunosuppression was induced with H2O2 and the production of immunoglobulins was evaluated at the level of bone marrow and spleen. As shown in FIG. 9, the increase in the heavy and light chains of immunoglobulins can be seen in mice treated with said first composition compared to those treated with H2O2 alone .

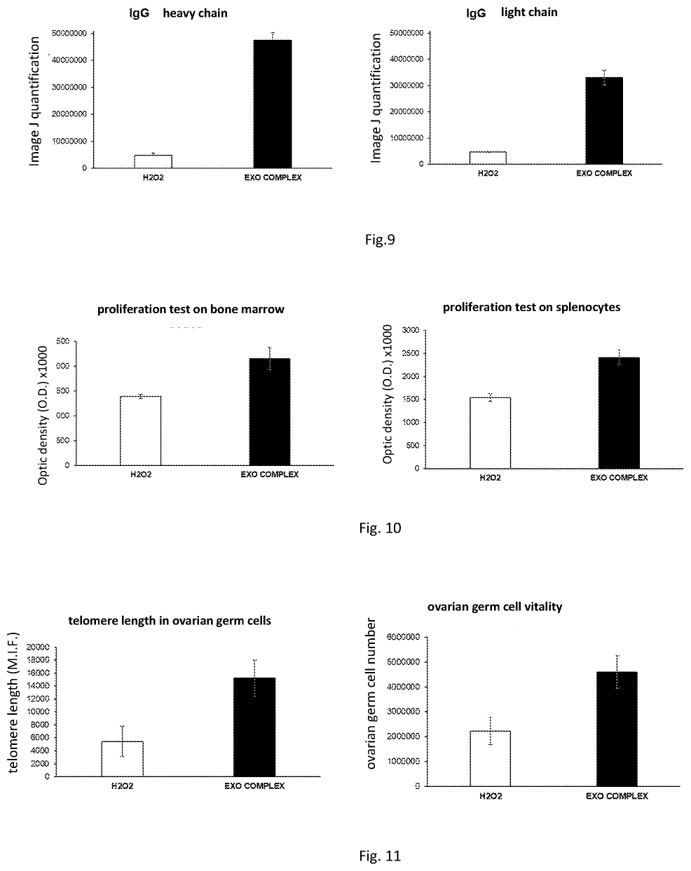

As shown in FIG. 10, in the same experiments the proliferation capacity in the bone marrow and spleen of the mice treated with said first composition was evaluated. These experiments demonstrated a significant increase in the proliferation capacity of the cells obtained from both organs of the mice treated with this first composition.

A second composition, as demonstrated by experimental tests, comprises a blend capable of inducing anti-aging effects developed on the basis of the properties of the individual fruits.

The second composition includes a blend of fruits derived from certified Italian biological cultivations, extracted in the percentages shown in Table 5.

TABLE 5
COMPOSITION MIX

| | |
|---|---|
| Citrus Lemon | 23-27% |
| Citrus Sinensis | 23-27% |
| Citrus Sinensis | 32-38% |
| (Magnolopsida | |
| cultivar) | |
| Carica Papaya Linn | 4-6% |
| Citrus Bergamia | 4-6% |
| Mangifera Indica | 4-6% |

Concentrated nanovesicles are obtained from such fruit blend, according to the previously indicated extraction procedure.

The choice of the daily dose is defined for example as: 6×10⁹ nanovesicles. The quantification of the antioxidants contained in the 6×10⁹ nanovesicles dose is shown in Table 6 below.

TABLE 6
Quantification of antioxidants per dose of
6 × 10⁹ nanovesicles

| | | |
|---|---|---|
| Total antioxidant | 1777.0 ± 150 | mM |
| capacity | | |
| Superoxide dismutase | 527.0 ± 14 | U/ml |
| type I | | |
| Catalase | 919.9 ± 45 | mU/ml |
| Glutathione | 551.9 ± 12 | μM |
| Ascorbic acid | 4.61 ± 0.01 | μg |

The effect of said second anti-aging composition was evaluated at the molecular level by measuring the length of telomeres on ovarian germ cells obtained from prematurely aged mice following treatment with H2O2 and treated or not treated with such second composition.

As shown in FIG. 11, telomere length was tripled in mice treated with the second composition compared to untreated mice. This data is accompanied by a doubling of the number of cells in the organs examined.

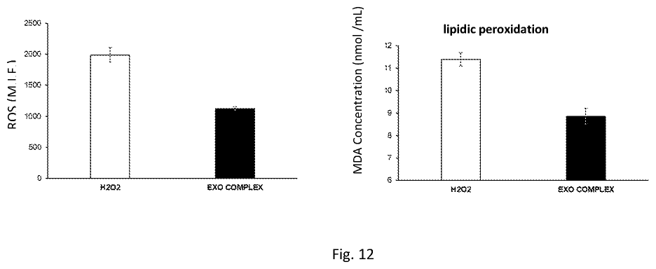

As shown in FIG. 12, in the same experiments the levels of oxidizing substances (ROS) and lipid peroxidation present in the blood of mice treated and not treated with said second anti-aging composition were evaluated. It is possible to notice a drastic reduction of these values as evidence of the powerful anti-oxidant effect of said second composition. A third composition, as demonstrated by experimental tests, includes a blend capable of accelerating the wound repair process on the basis of the properties of the individual fruits.

The third composition includes a blend of fruits derived from certified Italian biological cultivations, extracted in the percentages shown in Table 7.

TABLE 7
COMPOSITION MIX

| | |
|---|---|
| Malus domestica | 27-33% |
| (MADONIE cultivar) | |
| Capsicum Annum L. | 18-22% |
| Citrus Reticulata | 27-33% |
| Carica Papaya Linn | 18-22% |

Concentrated nanovesicles are obtained from such fruit blend, according to the previously indicated extraction procedure.

The choice of the daily dose is defined for example as: 5×10 10 nanovesicles.

The quantification of the antioxidants contained in the 5×10 10 nanovesicles dose is shown in Table 8 below.

TABLE 8
Quantification of antioxidants per dose of
5 × 10¹⁰ nanovesicles

| | | |
|---|---|---|
| Total antioxidant | 42722 ± 11912 | mM |
| capacity | | |
| Superoxide dismutase | 369622 ± 3015 | U/ml |
| type I | | |
| Catalase | 24959 ± 1145 | mU/ml |
| Glutathione | 590 ± 17 | μM |
| Ascorbic acid | 96.4 ± 0.07 | μg |

The regenerating effect of said third composition was initially evaluated on the spontaneous mortality of human fibroblasts and keratinocytes kept in culture in the presence or absence of such third composition.

As shown in FIG. 5, in both cases the presence of the third composition halves the number of dead cells in the treated cases compared to the control.

As shown in FIG. 6, the presence of the third composition accelerates the wound repair process in human keratinocyte cultures in which concurrently a solution was continuously produced and the margin rejoining time (wound healing test) was evaluated. The addition of the third composition was compared to that of commercial ascorbic acid. The results showed that in cultures treated with the third composition as early as 24 h, the wound margins were almost completely repaired by the proliferating cells.

As shown in FIG. 7, the presence of the third composition increases the production of collagen in the same cultures. The results showed a net increase in the production of collagen in the cultures treated with said third composition. Even if at least one exemplary embodiment has been presented in the summary and in the detailed description, it is to be understood that there are a large number of variants within the scope of the invention. Furthermore, it must be understood that the presented embodiment or embodiments are only examples which are not intended to limit in any way the scope of protection of the invention or its application or configurations. On the contrary, the brief and the detailed description provide the technician skilled in the art with a convenient guide for implementing at least one exemplary embodiment, it being clear that numerous variants can be made in the function and assembly of the elements described herein, without departing from the scope of protection of the invention as established by the attached claims and by their technical-legal equivalents.

1. Nanovesicles derived from plants of biological origin, comprising a lipid membrane and characterized in that they contain at least one bio-available antioxidant substance within the lipid membrane.

2. Nanovesicles according to claim 1, wherein the total antioxidant capacity of the at least one bio- available antioxidant substance for a dose equal to 5×10¹⁰ nanovesicles is greater than 1600 mM.

3. Nanovesicles according to claim 1, wherein said at least one bio-available antioxidant substance is Ascorbic Acid.

4. Nanovesicles according to claim 1, wherein its size is comprised between 80 nm and 200 nm.

5. Nanovesicles according to claim 1, obtained from at least one of the organic plants comprising Citrus paradisi, Citrus lemon (L.) Citrus reticulata, Citrus bergamia, Actinidia chinensis, Mangifera indica, Carica papaya linn, Citrus sinensis, Malus domestica.

6. Nanovesicles according to claim 1, to be used as a nutraceutical.

7. Nanovesicles according to claim 1, to be used as a cosmetic.

8. Nanovesicles according to claim 1, to be used as a regenerative.

9. A first composition of nanovesicles according to claim 1, wherein for a dose equal to 5×10¹⁰ nanovesicles, the antioxidant capacity is equal to 1739.0±130 mW.

10. The first composition according to claim 9, wherein the nanovesicles are extracted from a mixture comprising:
Citrus lemon in a percentage between 18% and 22%,
Citrus sinensis in a percentage between 18% and 22%,
Citrus sinensis (Magnolopsida cultivar) in a percentage between 9% and 11%,
Carica papaya linn in a percentage between 18% and 22%,
Mangifera indica in a percentage between 9% and 11%,
Actinidia chinensi in a percentage between 18% and 224%.

11. The first composition according to claim 9, further comprising the following bio-available antioxidant substances:
Superoxide Dismutase type I in an amount equal to 556.8±13 U/ml,
Catalase in an amount equal to 1713.3±30 m U/ml,
Glutathione in an amount equal to 549.4±15 pM and
Ascorbic Acid in an amount equal to 32.3±0.1 pg.

12. A use of the first composition according to claims 9, to induce immunostimulation and immunonutrition effects.

13. A second composition of nanovesicles according to claim 1, wherein for a dose equal to 6×10⁹ nanovesicles, the antioxidant capacity is equal to 1777.0±150 mM.

14. The second composition according to claim 13, wherein the nanovesicles are extracted from a mixture comprising:
Citrus lemon in a percentage between 23% and 27%,
Citrus sinensis in a percentage between 23% and 27%,
Citrus sinensis (Magnolopsida cultivar) in a percentage between 32% and 38%,
Citrus paradisi in a percentage between 4% and 6%,
Citrus bergamia in a percentage between 4% and 6%
Mangifera indica in a percentage between 4% and 6%.

15. The second composition according to claim, further comprising the following bio-available antioxidant substances:
Superoxide Dismutase type I in an amount equal to 527.0±14 U/ml,
Catalase in an amount equal to 919.9±45 mU/ml,
Glutathione in an amount equal to 551.9±12 pM and Ascorbic Acid in an amount equal to 4.61±0.01 μg.

16. A use of the second composition according to claims 13 to induce anti-aging effects.

17. A third composition of nanovesicles according to claim 1, wherein for a dose equal to 5×10¹⁰ nanovesicles the antioxidant capacity is equal to 42722±11912 mM.

18. The third composition according to claim 17, wherein the nanovesicles are extracted from a mixture comprising:
Malus domestica (DELLE MADONIE cultivar) in a percentage between 27% and 33%,
Capsicum annum L. in a percentage between 18% and 22%,
Citrus reticulata in a percentage between 27% and 33%,
Carica papaya linn in a percentage between 18% and 22%.

19. The third composition according to claim 17, further comprising the following bio-available antioxidant substances:
Superoxide Dismutase type I in an amount equal to 369622±3015 U/ml,
Catalase in an amount equal to 24959±1145 mU/ml,
Glutathione in an amount equal to 590±17 μM and
ascorbic acid in an amount equal to 96.4 ±0.07 pg.

20. Use of the third composition according to claim 17 to accelerate the wound repair process.
